(19) United States
(12) Patent Application Publication
(10) Pub. No.: US 2005/0008606 A1
(43) Pub. Date: Jan. 13, 2005

(54) Personal care composition

(76) Inventors: Emma Pawson, Cheshire (GB); Caroline Birrane, Manchester (GB); Michael David Cooper, Macclesfield (GB); David George Bishop, Manchester (GB); Emma Baxter, Halifax (GB)

Correspondence Address: SALTER & MICHAELSON;THE HERITAGE BUILDING, 321 SOUTH MAIN STREET, PROVIDENCE, RI, 029037128, US

(21) Appl. No.: 10/852,666

(22) Filed: May 24, 2004

(30) Foreign Application Priority Data
May 23, 2003 (GB) 0311937.7

(51) Int. Cl. A61K 7/06; A61K 7/11

(52) U.S. Cl. 424/70.17; 424/78.27

(57) ABSTRACT

An antibacterial personal care composition comprising at least one surfactant and at least one pH modifier said composition having a pH in the range of from 3.5 to 6.0. The composition further comprises at least one cationic polymer such that the composition is selectively active against transient bacteria relative to resident bacteria.

The present invention relates to an antibacterial personal care composition which finds utility in areas such as personal washing, hand washing and body washing.

As referred to herein, a personal care composition is a composition for cleaning the skin and/or hair.

There is a consumer demand for cleansing compositions which can assist in the removal of organisms, and in particular bacteria from the skin. These cleansers are preferred as in addition to cleansing the skin they can also remove bacteria from the skin. Prior art cleansers achieve this antibacterial efficacy by employing an antibacterial agent. However, the use of these antibacterial agents presents a problem in that they not only remove transient bacteria from the skin, but they also remove resident bacteria. As referred to herein, transient bacteria are non-resident bacteria which can spread between individuals and result in disease and other health problems. An example of a transient bacteria found on the skin is the gram negative Pseudomonas stutzeri. Whilst it is beneficial to remove transient bacteria from human skin it is not desirable to remove all resident bacteria from human skin. As referred to herein, resident bacteria are those which exist naturally in human intestines and on human skin. The role of resident bacteria is thought to be beneficial to us in destroying more dangerous transient bacteria. In fact many of these organisms enjoy a symbiotic relationship with us. An example of a resident bacteria found on skin is gram positive Micrococcus luteus.

In addition to these problems, some well known antibacterial agents can also have an unpleasant odour and detrimental effects on the environment. Furthermore, the pH of many compositions comprising these antibacterial agents can be higher than the skin's natural pH i.e. 4.5 to 5.5.

WO 95/32705 discloses an antimicrobial liquid cleansing formulation that attempts to address this pH issue whilst maintaining the antimicrobial effectiveness and mildness of the composition. This is achieved by buffering the pH of the composition to no more than 5.5, by using acid as the buffering agent for example hexanoic acid. However, hexanoic acid has a foul disagreeable odour and is consequently not suitable for use in personal care washing compositions. The substitution of hexanoic acid by other acids suggested in WO 95/32705 has been found not to provide good antibacterial efficacy for a broad range of bacteria. Moreover, WO 95/32705 does not address the problem of destroying transient bacteria whilst leaving resident bacteria substantially unaffected.

WO 98/55093 describes a mild rinse-off antimicrobial liquid cleansing composition which contains an antimicrobial active agent. WO 98/55093 does not address the problem of destroying transient bacteria whilst leaving resident bacteria substantially unaffected.

GB 2378186 describes an antimicrobial liquid cleansing formulation comprising a blend of surfactants, acid or acid anhydrides, phospholipids and water, the formulation having a pH from 3.5 to 5.5.

Once again the formulation described in GB 2378186 does not address the problem of destroying transient bacterial whilst leaving resident bacteria substantially unaffected.

Therefore, it is desirable to produce an antibacterial personal care composition which is active against transient bacteria yet substantially inactive against resident bacteria and is mild upon the skin.

According to the present invention there is provided an antibacterial personal care composition comprising at least one surfactant, at least one pH modifier and at least one cationic polymer, the personal care composition having a pH in the range from 3.5 to 6.0, wherein the composition effects a median log reduction in viable counts of greater than 2 against transient bacteria as determined by the suspension test described herein, and wherein a median log reduction in activity against resident bacteria is at least 0.5 less than the result for transient bacteria as determined by the suspension test defined hereinafter.

### Suspension Test Protocol

Dilution Medium for Bacteria

| | | |
|---|---|---|
| Tryptone water: | Tryptone (Oxoid L42) | 1.0 g |
| | Sodium Chloride | 8.5 g |
| | Distilled water | 1000 ml |

Adjust pH to 7.2+/−0.2 at 20° C. before autoclaving.

Inactivating Medium

| | |
|---|---|
| Tryptone Soya Broth (pH 7.2) + | 10.0% Tween 80 |
| | 3.0% Lecithin |
| | 0.1% L-Histidine |

After autoclaving add 1 ml 0.5% Sodium Sulphate solution (in distilled water, sterilised by filtration) to 8 ml Inactivating Medium
Bacterial Test Suspensions
1. Grow organism overnight at 37° C. on TSA to obtain fresh colonies (five days for propionibacteria on Wilkins Chalgren agar).
2. Resuspend colonies of the cells in the diluent using a vortex mixer to match a 0.5 McFarland standard (1-3×10⁸ cells/ml).
3. Maintain the suspension in a water bath at 20° C.±1° C. and use within 2 hours.
4. In order to count the bacterial test suspension prepare 10⁻⁴ and 10⁻⁶ dilutions of the suspension above using diluent.
5. Take 0.1 ml of each dilution, in duplicate, and spread each 0.1 ml sample into separate TSA plates (Wilkins Chalgren for P.acnes) using a sterile spreader.
6. Incubate plates at 37° C. overnight (7 days for P. acnes)
7. Count colonies in accordance with instructions below and calculate the cfu/ml in the test suspension. Note the T0 value will be 10× less than this number due to dilution in the test solution.
   Test Protocol
   Range Tested 8%-90% (Dilution)
1. Equilibrate all reagents (sample test solution, water, bacterial test suspension, neutraliser) to a temperature of 20° C.±1° C. using a water bath ensuring all reagents are stable at this temperature.
2. Pipette 9.0 ml of one of the sample test solutions into a container of suitable capacity (90% solution). To produce an 8% solution 0.8 ml of sample was diluted to 9 ml with sterile distilled water.
3. Add 1.0 ml of one of the bacteria test suspensions containing 1.5×10⁸ to 5×10⁸ cfu/ml.
4. Immediately start the stopwatch, mix and place the container in a water bath controlled at 20° C.±1° C.
5. Determine the activity of the sample at a contact time of 1 min±10 s.
6. Just before end of chosen contact time, mix.
7. At the chosen contact time, pipette 1.0 ml of the test mixture into a tube containing 8.0 ml neutraliser and 1.0 ml water.
8. Mix and place in a water bath controlled at 20° C.±1° C.
9. Allow to neutralise for 5 min±10 seconds.
10. After neutralisation, immediately take a sample of 0.1 ml of neutralised mixture and dilute to 10⁻⁴ in neutralization solution. Transfer in duplicate 0.1 ml of required dilutions on to separate TSA plates (or Wilkins Chalgren plate for P. acnes). and spread using a sterile spreader.
11. Repeat steps 2 to 10 of the procedure using other sample test solutions.
12. Incubate the plates overnight at 37° C. (7 days anerobically for P. acnes)
13. Count the plates as referred to below.
   Counting of Bacteria
1. Incubate the bacterial spread plates prepared as above at 36° C.±1° C. for 24 hours or 7 days anerobically for P. acnes (disregard any plates which are not countable).
2. Count the plates which contain between 30 and 300 colonies and determine the number of colony forming units for each plate.
3. Calculate the number of cfu/ml in the test suspension and time points
   Cfu/ml in test=Average count×10×dilution factor (ie×1000 for a count on a 10⁻³ plate).
   To calculate log reduction for the suspension test
   Log₁₀ cfu/ml of test suspension (T0)−Log₁₀ cfu/ml of suspension after 1 min
   Note the cfu/ml value calculated for the original test suspension is 10×the amount (T0) in the test solution (1 ml test suspension added to 9 ml test solution).
   Unless otherwise stated all reagents are prepared in accordance with EN1040:1997 and all definitions are as referred to in EN1040:1997.

As referred to herein, a typical transient bacteria is the gram negative bacteria Pseudomonas stuzeri whilst a typical resident bacteria is the gram positive bacteria Micrococcus luteus.

Advantageously, the use of the composition of the present invention leaves resident bacteria substantially unaffected. Furthermore, the composition of the present invention is sufficiently mild that the health of the skin is maintained.

It is thought that the bacterial selectivity of the composition of the present invention arises from a combination of the low pH of the composition and the cationic polymer. It is thought that since the low pH of the composition maintains or even promotes the health of the skin, favourable skin conditions arise which enables the resident bacteria to thrive. In addition to this, when the cationic polymer is deposited onto the skin it leaves a protective coating which helps prevent transient bacteria settling onto the skin, and hence preventing bacteria growth.

Suitable surfactants, which may be used alone or in combination, include anionic, cationic, non-ionic or amphoteric surfactants.

Specific examples include alkali metal alkyl ether sulphates, alkyl sulphates, alkyl ether sulphonates, sulphosuccinates, acyl glutamates, alkyl polyglucosides, isethionates, carboxylates, soaps, ethoxylated and non ethoxylated metal alkyl sulphamates, sultanes, taurates, sarcosinates, sulphonates, ether carboxylates, glycinates, quaternary ammonium compounds, polysorbates, sugar esters, alkyl phosphates, propionates, amino acid surfactants, glycides, alkanolamides alkylbetaines, amidopropyl betaines and amidopropyl sultaines.

The surfactant preferably constitutes from between 0.1 and 60% by weight of the total composition.

Suitable cationic polymers, which may be used alone or in combination, include guar hydroxypropyltrimonium chloride or a polymer consisting of acrylic acid, diallyl dimethyl ammonium chloride or acrylamide. Other cationic polymers that are suitable for use either alone or in combination include quaternised cellulose ethers, copolymers of dimethyl aminoethylmethacrylate and acrylamide, quaternized vinyl pyrrolidone acrylate or methacrylate copolymers of amino alcohol and cationic phospholipids. The preferred cationic polymer comprises an amphoteric terpolymer.

The cationic polymer is preferably water-soluble.

The cationic polymer preferably constitutes from 0.01 to 2.0% by weight of the total cleansing composition.

In addition the cationic polymer provides moisturising properties, lubricity, and rich foam properties to the composition. Furthermore, the cationic polymer may also result in a smooth skin after feel.

A pH modifier is used to adjust the pH of the composition. The said pH modifier may be acidic or alkaline.

Suitable pH modifiers, which may be used alone or in combination, include any of the following: mineral acids such as hydrochloric acid, phosphoric acid and sulphuric acid, organic acids such as benzoic acid, citric acid, lactic acid, maleic acid, malic acid, tartaric acid, adipic acid, gluconic acid and their salts and bases such as sodium hydroxide and potassium hydroxide.

The pH modifier is preferably citric acid or a metal salt thereof such as sodium citrate.

The amount of pH modifier used in the composition is dictated by the pH of the final product, but is typically 0.01-1.0% by weight of the total cleansing composition.

The pH of the composition is preferably in the range 4.0 to 5.0. As referred to herein the pH described is that of the neat product taken at 20° C.

The composition of the present invention may comprise one or more additional ingredients including skin feel agents, antibacterial agents, colour and fragrances.

According to the second aspect of the present invention there is provided a method for the preparation of an antibacterial personal care composition as hereinbefore defined comprising the steps of: adding to water a mixture comprising at least one surfactant and at least one cationic polymer and adding a pH modifier to the said mixture to adjust the pH of the composition to between 3.5 and 6.0.

Any additional ingredients may be added prior to the adjustment of the pH.

The present invention will now be described by way of example only and with reference to the following examples and drawings:

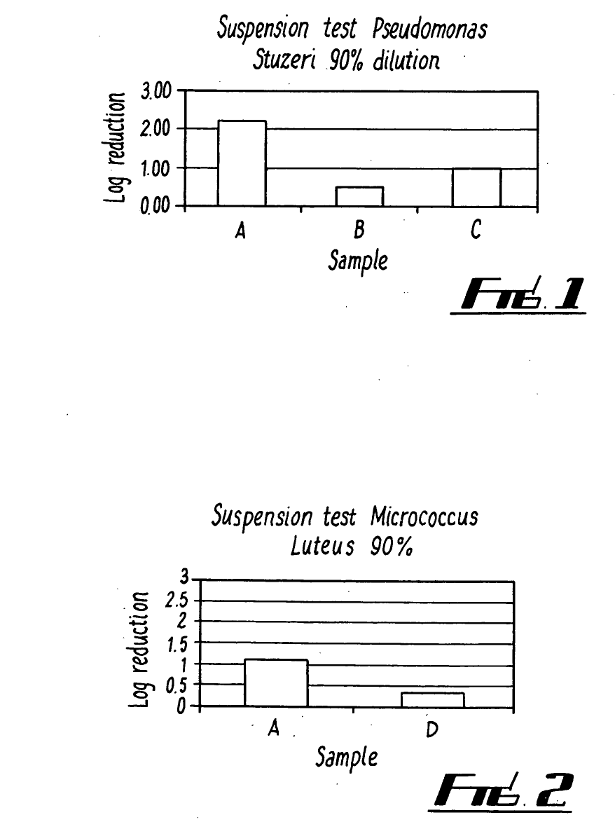

FIG. 1 is a bar chart showing the activity of a 90% aqueous dilution of one embodiment of the composition of the present invention against a transient bacteria, Pseudomonas stuzeri; and

FIG. 2 is a bar chart showing the reduced activity of a 90% aqueous dilution of one embodiment of the composition of the present invention against a resident bacteria. Micrococcus luteus.

### EXAMPLE

Liquid Hand Soap

It is common for antibacterial liquid hand soaps to be effective at reducing the number of bacteria when tested using an industry recognised in-vitro suspension test. However this test quantitatively assesses the amount the bacteria is reduced by and illustrates this value as either a percentage reduction or log reduction. Using the in-vitro test mentioned herein, the formulation shown in the following example reduces the number of transient bacteria whilst the resident bacteria are substantially unaffected. This effect can be described as “maintaining the skins natural micro flora”. It is generally thought a score of log 2 or more means that the product is effective at killing bacterial (99% kill level).

The following examples A to D were prepared:

| | | Cationic | | Anionic | Nonionic | Amphoteric | pH | pH |
|---|---|---|---|---|---|---|---|---|
| | | Polymer | Cationic | surfactant | Surfactant | Surfactant | modifier | modifer |
| Example | pH | level % | Polymer type | level % | level | level % | level | type |
| A | 4.2 | 0.15 | Guar | 6 | 3 | 2-3 | 0.7 | Citric |
| | | | Hydroxypropyl- | | | | | acid |
| | | | trimonium | | | | | |
| | | | Chloride | | | | | |
| B | 4.1 | 0.0 | — | 6 | 3 | 2-3 | 0.7 | Citric |
| | | | | | | | | Acid |
| C | 6.9 | 0.15 | Guar | 6 | 3 | 2-3 | 0.05 | Citric |
| | | | Hydroxypropyl- | | | | | acid |
| | | | trimonium | | | | | |
| | | | Chloride | | | | | |
| D | 4.2 | 0.5 | Polyquaternium- | 6 | 1 | 2-3 | 0.5 | Ciric |
| | | | 39 | | | | | acid |

To test this theory suspension testing has been conducted using the most common transient bacteria found on hands (Pseudomonas stutzeri). It is generally thought that at 90% and 8% dilution a result above log 2 equates to effective bacterial kill. The following samples were tested:
A. a sample of liquid soap at pH 4.2+cationic polymer
B. a sample at pH 4.2 without cationic polymer
C. a sample at pH 7.0 with cationic polymer.

The results, shown in FIG. 1, show that the combination of low pH and cationic polymer unexpectedly gives a boosted score against transient bacteria i.e. above log 2.

As depicted in FIG. 2, sample A was also tested against resident bacteria to check that the bacteria remained substantially unaffected. Micrococcus luteus bacteria was used, this is a common resident bacteria found on hands.

The cationic polymer of sample A was substituted with another cationic polymer (i.e. an amphoteric terpolymer) and tested against the same resident bacteria. This sample is referred to as sample D. The results, shown in FIG. 2, show that with two different types of cationic polymers the effect on resident bacteria is minimal showing that the effect is not simply attributed to a certain type of cationic polymer. Dilutions were 90% or 8%.

A further series of tests, again using modified EN1040 as described herein, were conducted on examples as outlined in tables 1 and 2.

TABLE 1

| | | Cationic | | Anionic | Nonionic | Amphoteric | pH | pH |
|---|---|---|---|---|---|---|---|---|
| | | Polymer | Cationic Polymer | surfactant | Surfactant | Surfactant | modifier | modifer |
| Example | pH | level % | type | level % | level % | level % | level | type |
| E | 4.2 | 0.5 | Polyquaternium- | 6 | 1 | 2-3 | 0.5 | Citric |
| | | | 39 | | | | | acid |
| F | 7.0 | 0.5 | Polyquaternium- | 6 | 0 | 2-3 | 0.04 | NaOH |
| | | | 39 | | | | | |
| G | 4.0 | 0.9 | Cocamidopropyl | 6 | 0 | 2-3 | 0.7 | Citric |
| | | | polyquaternium- | | | | | acid |
| | | | 39 | | | | | |
| H | 7.0 | 0.5 | Polyquaternium- | 6 | 0 | 2-3 | 0.23 | Ciric |
| | | | 39 | | | | | acid |
| | | | | | | | | NaOH |

TABLE 2

| | | | | | | | | Median |
|---|---|---|---|---|---|---|---|---|
| | | | | | | | | log |
| | | | | | | | | reduction |
| | | | | | | | | in |
| | | | | | | | | activity |
| | | | | | | | | against |
| | | | | | | | | skin |
| | | | | Log | Log | Log | Log | resident |
| | | Log | Log | Reduction | Reduction | Reduction | Reduction | bacteria |
| | | reduction | Reduction | Listeria | Staphlococcus | Staphlococcus | P Acnes | cf results |
| | | E Coli | P Stutzeri | Moncytogens | Aureus | epidermidis | Skin | from |
| Example | Dilution % | Transient | Transient | Transient | Transient | Skin Resident | Resident | transient |
| E | 90 | NT | 7.08 | NT | NT | 2.63 | NT | 4.45 |
| F | 90 | 1.8 | NT | 1.6 | 0.7 | 0.8 | 1.0 | 0.46 |
| G | 8 | 7.0 | NT | NT | 7.0 | NT | NT | — |
| H | 8 | 0 | NT | NT | 0.1 | NT | NT | — |

NT = not tested

Examples E and F show the importance of the pH of the composition on the efficacy of the composition. Example F has a pH of 7.0 i.e. outside the scope of the present invention and, as can be seen from table 2, does not provide a log reduction in the transient bacteria of>2 and nor does it provide a median log reduction in activity against resident bacteria being at least 0.5 less than the result for transient bacteria. In contrast to this the pH of example E is 4.2 i.e. within the scope of the present invention and example E does provide a median log reduction in activity against resident bacteria of at least 0.5 less that the result for transient bacteria.

This is further supported by the results obtained for examples G and H.

It is of course to be understood that the invention is not intended to be restricted to the details of the above embodiments which is described by way of example only.

1. According to the present invention there is provided an antibacterial personal care composition comprising at least one surfactant, at least one pH modifier and at least one cationic polymer, the personal care composition having a pH in the range from 3.5 to 6.0, wherein the composition effects a median log reduction in viable counts of greater than 2 against transient bacteria as determined by the suspension test defined herein, and wherein a median log reduction in activity against resident bacteria is at least 0.5 less than the result for transient bacteria as determined by the suspension test defined herein.

2. An antibacterial personal care composition according to claim 1, wherein the surfactant constitutes from 0.1 to 60% by weight of the total composition.

3. An antibacterial personal care composition according to claim 1, wherein the cationic polymer comprises an amphoteric terpolymer.

4. An antibacterial personal care composition according to claim 1, wherein the cationic polymer is water-soluble.

5. An antibacterial personal care composition according to claim 1, wherein the cationic polymer constitutes from 0.01 to 2.0% by weight of the total composition.

6. An antibacterial personal care composition according to claim 1, wherein the pH modifier, comprises any of the following either alone or in combination: —include any of the following: mineral acid, organic acid and an alkali.

7. An antibacterial personal care composition according to claim 1, wherein the pH modifier comprises at least one of citric acid and a metal salt thereof.

8. An antibacterial personal care composition according to claim 1, wherein the pH modifier constitutes from 0.01 to 1.0% by weight of the total composition.

9. An antibacterial personal care composition according to claim 1, wherein the composition has a pH in the range from 4.0 to 5.0.

10. A method for the preparation of an antibacterial personal care composition as hereinbefore defined comprising the steps of: adding water to a mixture comprising at least one surfactant and at least one cationic polymer and adding a pH modifier to the said mixture to adjust the pH of the composition to from 3.5 to 6.0.
